(19) United States
(12) Patent Application Publication
(10) Pub. No.: US 2022/0177919 A1
(43) Pub. Date: Jun. 9, 2022

(54) CYBB LENTIVIRAL VECTOR, LENTIVIRAL VECTOR-TRANSDUCED STEM CELL, AND PREPARATION METHOD AND APPLICATION THEREOF

(71) Applicant: BEIJING MEIKANG GENO-IMMUNE BIOTECHNOLOGY CO., LTD., Beijing (CN)

(72) Inventor: Shuo Wang, Beijing (CN)

(21) Appl. No.: 17/604,360

(22) PCT Filed: Apr. 17, 2020
(86) PCT No.: PCT/CN2020/085243

§ 371 Date: Oct. 15, 2021

(30) Foreign Application Priority Data
Apr. 17, 2019 (CN) 201910310154.3

(51) Int. Cl. C12N 15/86; C12N 7/00; A61K 38/44; C12N 9/02

(52) U.S. Cl. CPC C12N 15/86; C12N 7/00; A61K 38/44; C12N 2740/15043; C12Y 106/03; C12N 2740/15032; C12N 2740/15052; C12N 9/0036

(57) ABSTRACT

Provided are a CYBB lentiviral vector, a lentiviral vector-transduced stem cell, a preparation method and application thereof. The lentiviral vector includes a hEF 1α promoter and CYBB that are organized in tandem. The lentiviral vector carries the CYBB gene which under the initiation of the hEF 1α promoter, and expresses the carried CYBB gene in differentiated or undifferentiated stem cells. Stem cells serve as a delivery vector.

### TECHNICAL FIELD

The present disclosure belongs to the technical field of genetic engineering, and relates to a lentiviral vector, a lentiviral vector-transduced stem cell, and a preparation method and application thereof, and in particular to a CYBB lentiviral vector, a lentiviral vector-transduced stem cell, and a preparation method and application thereof.

### BACKGROUND

Chronic granulomatous disease (CGD) is a hereditary and primary immunodeficiency disease caused by loss of function of NADPH oxidase in neutrophilic granulocytes and monocytes. It is characterized by repeated infection, inflammation and autoimmunity in patients. NADPH oxidase consists of p47phox, p40phox, p67phox, and gp91-phox, and loss of function of any part will cause CGD. Most patients are X-chromosome sex-linked recessive inherited due to gp91-phox mutation, and a few are autosomal recessive inherited. Patients often have a family history, and the symptoms are often found in children. Patients with mutation in the gp91-phox subunit gene of cytochrome b are the most common, accounting for about 65% of the total number of patients. The mutated gp91 gene is named CYBB (MIM306400) which contains 13 exons and is located on X chromosome xp21.1, occupying approximately 30 kb.

The NADPH oxidase complex consists of a membrane-binding protein and a cytoplasmic protein. They have a synergistic effect during phagocyte activation and assist the production of reactive oxygen species (ROS) to kill bacteria and fungi. In general, normal granulocytes phagocytize bacteria and de-granulate to produce hydrogen peroxide and release new ecological oxygen which oxidizes iodine and chlorine compounds to free iodine and chlorine, thereby achieving a complete hydrogen peroxide-peroxidase-iodide ion bactericidal system. However, CGD patients cannot produce hydrogen peroxide and cannot exert bactericidal effects in vivo due to NADPH oxidase deficiency, such that purulent infections occurs repeatedly in various parts of the body, which leads to purulent lymphadenitis, rhinitis, and sinusitis, and purulent inflammation in pericardium, lung, liver and nerve system. Patients may exhibit cutaneous granulomas, eczema dermatitis, hepatomegaly and splenomegaly, and granulomas formed by histiocytes containing pigment lipids in affected organs. Most of the patients die from severe infections at a young age. [Reference 7]

Currently, the only way to completely cure CGD is hematopoietic stem cell transplantation. However, finding a suitable donor is just one of the problems. The chemotherapy dose of transplant pretreatment, the infection status of the patient at the time of transplantation, the control of GVHD after transplantation, and the ability of CGD patients to rebuild the immune system after transplantation are the key factors that affect the success of the transplantation [Reference 8].

Since CGD is a disease caused by a single gene mutation, gene therapy is another potential treatment. At present, many studies in China and abroad have reported to gene therapy applications using viral vectors. However, different viral vectors or even different preparation methods of the same viral vector often have significantly different gene delivery efficiency, which directly affects the therapeutic effect of the gene therapy. At present, most cell and gene therapy methods for genetic diseases have an efficiency issue, and these methods are only applicable to blood stem cells, and the clinical effect is not as expected [Reference 8].

Phase I and II clinical trials in South Korea used retroviruses as vectors. Although no significant side effects were shown, genetically modified cells could not persist in patients for a long time [Reference 9]. Ravin et al. used CRISPR-Cas9 to repair mutant CYBB gene in CGD patients. However, the CRISPR-Cas9 system has targeting problems and potential safety hazards. Moreover, this method requires stringent conditions and high establishment costs, and results are unstable [Reference 10].

Lentiviral vector-mediated autologous stem cell gene therapy has been successfully applied to the treatment of diseases such as X-linked severe combined immunodeficiency (X-SCID), β-thalassemia, and sickle cell disease (SCD). Although the use of adenoviral vectors in gene therapy of hemophilia has been attempted since the 1990s, no animal experiment has reported positive results for lifelong continuous expression of coagulation factors. The difficulty of this method may be attributed to the immune response caused by the vector, the inability to express exogenous genes efficiently and continuously, and the inability to express exogenous genes in appropriate regions. In the past 10 years, many clinical gene therapy trials use gamma-oncoretroviral vectors whose viral characteristics include integration into the promoter region near oncogenes, and transgene silencing mechanism and thus have poor safety and lack of long term persistence.

Therefore, there is an urgent need for a viral vector that has high gene delivery efficiency and is suitable for targeting stem cells to improve the therapeutic effect for CGD.

### SUMMARY

In view of the shortcomings in the prior art, the present disclosure provides a CYBB lentiviral vector, a lentiviral vector-transduced stem cell, and a preparation method and application thereof. The hEF1α promoter in the lentiviral vector initiates the expression of CYBB that is the gene associated with CGD. The lentiviral vector has good safety and high gene transfer efficiency, thereby laying a foundation for improving the therapeutic effect for CGD.

To achieve this, the present disclosure adopts the following technical solutions:

In a first aspect, the present disclosure provides a lentiviral vector comprising a hEF1α promoter and CYBB that are organized in tandem.

In the present disclosure, under the initiation of the hEF1α promoter, the lentiviral vector carrying the CYBB gene achieves efficient gene delivery while ensuring safety, which is beneficial to increase the expression amount of the CYBB gene in transgenic cells.

Preferably, the hEF1α promoter has a nucleic acid sequence as shown in SEQ ID NO.1.

The nucleic acid sequence of SEQ ID NO. 1 is:

gctagcatgcctaggtcgaccaattctcatgtttgacagcttatcatcg
ataagctttggagctaagccagcaatggtagagggaagattctgcacgt
cccttccaggcggcctccccgtcaccaccccccccaacccgccccgacc
ggagctgagagtaattcatacaaaaggactcgcccctgccttggggaat
cccagggaccgtcgttaaactcccactaacgtagaacccagagatcgct
gcgttcccgccccctcacccgcccgctctcgtcatcactgaggtggaga
agagcatgcgtgaggctccggtgcccgtcagtgggcagagcgcacatcg
cccacagtccccgagaagttggggggaggggtcggcaattgaaccggtg
cctagagaaagtggcgcggggtaaactgggaaagtgatgtcgtgtactg
gctccgccatacccgagggtgggggagaaccgtatataagtgcagtagt
cgccgtgaacgttctattcgcaacgggtttgccgccagaacacaggtaa
gtgccgtgtgtggttcccgcgggcctggcctctttacgggttatggccc
ttgcgtgccttgaattacttccacgcccctggctgcagtacgtgattct
tgatcccgagcttcgggttggaagtgggtgggagagttcgaggccttgc
gcttaaggagccccttcgcctcgtgcttgagttgaggcctggcctgggc
gctggggccgccgcgtgcgaatctggtggcaccttcgcgcctgtctcgc
tgctttcgataagtctctagccatttaaaatttttgatgacctgctgcg
acgctttttttctggcaagatagtcttgtaaatgcgggccaagatctgc
acactggtatttcggtttttggggccgcgggcggcgacggggcccgtgc
gtcccagcgcacatgttcggcgaggcggggcctgcgagcgcggccaccg
agaatcggacgggggtagtctcaagctggccggcctgctctggtgcctg
gcctcgcgccgccgtgtatcgccccgccctgggcggcaaggctggcccg
gtcggcaccagttgcgtgagcggaaagatggccgcttcccggccctgct
gcagggagctcaaaatggaggacgcggcgctcgggagagcgggcgggtg
agtcacccacacaaaggaaaagggcctttccgtcctcagccgtcgcttc
atgtgactccacggagtaccgggcgccgtccaggcacctcgattagttc
tcgagcttttggagtacgtcgtctttaggttggggggaggggttttatg
cgatggagtttccccacactgagtgggtggagactgaagttaggccagc
ttggcacttgatgtaattctccttggaatttgccctttttgagtttgga
tcttggttcattctcaagcctcagacagtggttcaaagtttttttcttc
catttcaggtgtcgtgaaaactctagagcggccgcggaggccgaattcc
gtcgaggatccacc.

Preferably, the CYBB has an amino acid sequence as shown in SEQ ID NO. 2.

The amino acid sequence of SEQ ID NO. 2 is:

MGNWAVNEGLSIFVILVWLGLNVFLFVWYYRVYDIPPKFFYTRKLLGSA
LALARAPAACLNFNCMLILLPVCRNLLSFLRGSSACCSTRVRRQLDRNL
TFHKMVAWMIALHSAIHTIAHLFNVEWCVNARVNNSDPYSVALSELGDR
QNESYLNFARKRIKNPEGGLYLAVTLLAGITGVVITLCLILIITSSTKT
IRRSYFEVFWYTHHLFVIFFIGLAIHGAERIVRGQTAESLAVHNITVCE
QKISEWGKIKECPIPQFAGNPPMTWKWIVGPMFLYLCERLVRFWRSQQK
VVITKVVTHPFKTIELQMKKKGFKMEVGQYIFVKCPKVSKLEWHPFTLT
SAPEEDFFSIHIRIVGDWTEGLFNACGCDKQEFQDAWKLPKIAVDGPFG
TASEDVFSYEVVMLVGAGIGVTPFASILKSVWYKYCNNATNLKLKKIYF
YWLCRDTHAFEWFADLLQLLESQMQERNNAGFLSYNIYLTGWDESQANH
FAVHHDEEKDVITGLKQKTLYGRPNWDNEFKTIASQHPNTRIGVFLCGP
EALAETLSKQSISNSESGPRGVHFIFNKENF.

Preferably, the CYBB has a nucleic acid sequence as shown in SEQ ID NO. 3.

The nucleic acid sequence of SEQ ID NO. 3 is:

atggggaactgggctgtgaatgaggggctctccatttttgtcattctgg
tttggctggggttgaacgtcttcctctttgtctggtattaccgggttta
tgatattccacctaagttcttttacacaagaaaacttcttgggtcagca
ctggcactggccagggcccctgcagcctgcctgaatttcaactgcatgc
tgattctcttgccagtctgtcgaaatctgctgtccttcctcaggggttc
cagtgcgtgctgctcaacaagagttcgaagacaactggacaggaatctc
acctttcataaaatggtggcatggatgattgcacttcactctgcgattc
acaccattgcacatctatttaatgtggaatggtgtgtgaatgcccgagt
caataattctgatccttattcagtagcactctctgaacttggagacagg
caaaatgaaagttatctcaattttgctcgaaagagaataaagaaccctg
aaggaggcctgtacctggctgtgaccctgttggcaggcatcactggagt
tgtcatcacgctgtgcctcatattaattatcacttcctccaccaaaacc
atccggaggtcttactttgaagtcttttggtacacacatcatctctttg
tgatcttcttcattggccttgccatccatggagctgaacgaattgtacg
tgggcagaccgcagagagtttggctgtgcataatataacagtagtgaac
aaaaaatctcagaatggggaaaaataaaggaatgcccaatccctcagtt
tgctggaaaccctcctatgacttggaaatggatagtgggtcccatgttt
ctgtatctctgtgagaggttggtgcggttttggcgatctcaacagaagg
tggtcatcaccaaggtggtcactcaccctttcaaaaccatcgagctaca
gatgaagaagaaggggttcaaaatggaagtgggacaatacatttttgtc
aagtgcccaaaggtgtccaagctggagtggcacccttttacactgacat
ccgcccctgaggaagacttctttagtatccatatccgcatcgttgggga
ctggacagaggggctgttcaatgcttgtggctgtgataagcaggagttt
caagatgcgtggaaactacctaagatagcggttgatgggccdttggcac
tgccagtgaagatgtgttcagctatgaggtggtgatgttagtgggagca
gggattggggtcacacccttcgcatccattctcaagtcagtctggtaca
aatattgcaataacgccaccaatctgaagctcaaaaagatctacttcta
ctggctgtgccgggacacacatgcctttgagtggtttgcagatctgctg
caactgctggagagccagatgcaggaaaggaacaatgccggcttcctca
gctacaacatctacctcactggctgggatgagtctcaggccaatcactt
tgctgtgcaccatgatgaggagaaagatgtgatcacaggcctgaaacaa
aagactttgtatggacggcccaactgggataatgaattcaagacaattg
caagtcaacaccctaataccagaataggagttttcctctgtggacctga
agccttggctgaaaccctgagtaaacaaagcatctccaactctgagtct
ggccctcggggagtgcatttcattttcaacaaggaaaacttctaa.

In a second aspect, the present disclosure provides a lentivirus which is introduced with the lentiviral vector as described in the first aspect.

In a third aspect, the present disclosure provides a host cell which is transduced with the lentivirus as described in the second aspect.

Preferably, the host cell includes a stem cell.

The stem cell of the present disclosure is used as a delivery vehicle to transport the lentiviral vector carrying the CYBB gene, thereby improving the expression efficiency and expression amount of the CYBB gene in differentiated or undifferentiated stem cells.

Preferably, the stem cell includes a hematopoietic stem cell.

According to the present disclosure, the hematopoietic stem cell is derived from blood or bone marrow and has the ability to differentiate into a series of somatic hematopoietic cells and the ability to renew various histiocytes. The hematopoietic stem cell of the present disclosure is used as a potential transport tool to carry the lentivirus containing the CYBB gene to achieve gene therapy for CGD.

In a fourth aspect, the present disclosure provides a method for preparing the host cell as described in the third aspect, which includes the following steps:

(1) constructing a lentiviral vector as described in the first aspect;

(2) performing lentivirus packaging by co-transducing the lentiviral vector obtained in step

(1) and a packaging plasmid into a mammalian cell, to obtain a lentivirus; and

(3) transforming the lentivirus obtained in step (2) into the genome of a host cell.

Preferably, the construction in step (1) is performed by inserting a hEF1α promoter and CYBB into TYF lentiviral vector through restriction enzyme digestion.

Preferably, the packaging plasmid in step (2) includes pNHP and pHEF-VSVG.

Preferably, the mammalian cell in step (2) includes a 293T cell.

Preferably, the method further comprises a step of purifying the lentivirus after step (2).

Preferably, the purification is performed by filtering and concentrating the lentivirus to high-titer virus.

In the present disclosure, the purification, filtration and concentration significantly improve the titer and concentration of the lentivirus.

Preferably, the host cell in step (3) includes a stem cell.

Preferably, the stem cell include a hematopoietic stem cell.

As a preferred technical solution, the present disclosure provides a method for preparing a host cell as described in the third aspect, which comprises the following steps:
(1) inserting a hEF1α promoter and CYBB into TYF lentiviral vector through restriction enzyme digestion to construct a lentiviral vector;
(2) performing lentivirus packaging by co-transfection of the lentiviral vector plasmid obtained in step (1) and packaging plasmids pNHP and pHEF-VSVG into a 293T cell to assemble a lentivirus, centrifuging the lentivirus at 1,000˜1100 g for 3˜5 minutes to remove cell debris, filtering the resulting supernatant with a 0.45˜0.5 μm low protein binding filter, centrifuging at 2000˜2500 g for 30˜40 min, shaking the filter tube, and centrifuging at 300˜400 g for 2˜5 min; and
(3) transduction of the lentivirus obtained in step (2) into the a hematopoietic stem cell.

In a fifth aspect, the present disclosure provides a pharmaceutical composition which includes any one or a combination of at least two of a group consisting of the lentiviral vector as described in the first aspect, the lentivirus as described in the second aspect, and the host cell as described in the third aspect.

Preferably, the pharmaceutical composition further includes any one or a combination of at least two of a group consisting of a pharmaceutically acceptable carrier, an excipient and a diluent.

The pharmaceutical composition of the present disclosure repairs the mutant CYBB gene of a CGD patient at genetic level, which is beneficial to the repair of the patient's autologous stem cells with potential for long-term stable treatment of CGD.

In a sixth aspect, the present disclosure provides the use of the lentiviral vector as described in the first aspect, the lentivirus as described in the second aspect, the host cell as described in the third aspect, or the pharmaceutical composition as described in the fifth aspect in the preparation of a medicament for treating a disease.

Preferably, the disease includes chronic granulomatous disease.

In a seventh aspect, the present disclosure provides a method for treating chronic granulomatous disease using the lentiviral vector as described in the first aspect, the lentivirus as described in the second aspect, the host cell as described in the third aspect, or the pharmaceutical composition as described in the fifth aspect.

According to the present disclosure, the method comprises the following steps:
(1′) mobilizing CD34 stem cells in a patient with granulocyte colony-stimulating factor, and collecting blood or bone marrow hematopoietic stem cells multiple times;
(2′) in laboratory, isolating CD34-positive cells from the bone marrow collected from the patient and culturing the same prior to infusion;
(3′) pre-treating the patient in a clinical setting prior to infusion;
(4′) in laboratory, performing gene transduction twice by infecting CD34 cells with the lentivirus carrying the CYBB gene, and culturing the cells prior to infusion;
(5′) on the day of infusion, washing and suspending the cells and then infusing the same back to the patient; and
(6′) after infusion to the patient, performing follow-up every week to collect the peripheral blood of the patient, and measure the oxidase function, immune cell ratio and gene copy number.

Preferably, the pretreatment in step (3′) is performed with busulfan 40 mg per kg of body weight and fludarabine 60 mg per m² of body surface area.

Preferably, the washing in step (5′) is performed by washing the cells twice with normal saline containing 1% of human serum protein.

Preferably, the suspension in step (5′) is performed with normal saline containing 2.5% of human serum albumin protein.

Compared with the prior art, the present disclosure has the following beneficial effects:
(1) The lentiviral vector of the present disclosure achieves safe and efficient expression of the carried CYBB gene in differentiated or undifferentiated stem cells under the initiation of the hEF1a promoter.
(2) In the present disclosure, lentivirus carrying CYBB gene is used to transduce stem cells to efficiently repair the patient's autologous stem cells, while the stem cells can serve as potential delivery vehicles to increase the expression amount of CYBB gene in differentiated transgenic cells.
(3) In the present disclosure, stem cells transduced with CYBB lentivirus are infused back into the patient such that the expression levels of oxidase in monocytes and neutrophilic granulocytes of patients are significantly increased, the number of neutrophilic granulocytes and monocytes expressing oxidase can be maintained at high levels, and the CYBB gene has good stability in peripheral blood cells, thereby achieving long-term stable treatment of chronic granulomatous disease with high safety. This method has potential for treating chronic granulomatous disease.

### BRIEF DESCRIPTION OF THE DRAWINGS

FIG. 1 is a schematic diagram showing the composition of the lentiviral vector;

FIG. 2 is a diagram showing a treatment procedure;

FIG. 3 (a) shows the analysis of peripheral blood from the patient's father who has a normal genotype, FIG. 3 (b) shows the analysis of peripheral blood from the patient's mother who has a heterozygous genotype, FIG. 3 (c) shows the analysis of peripheral blood from the patient before infusion, and FIG. 3 (d) shows the analysis of peripheral blood from the patient on day 28 after treatment;

FIG. 4 (a) shows the average fluorescence intensity multiple stained with rhodamine 123, and FIG. 4 (b) shows the proportion of neutrophilic granulocytes that emit fluorescence;

FIG. 5 (a) shows the number of neutrophilic granulocytes in CD45-positive cells after infusion, and FIG. 5 (b) shows the number of monocytes in CD45-positive cells after infusion;

FIG. 6 shows the change of CYBB gene copy number in peripheral blood from the patient after infusion;

FIG. 7 shows CT scan images of the lung of the patient before and after infusion.

### DETAILED DESCRIPTION

In order to further illustrate the technical measures adopted by the present application and the effects thereof, the technical solutions of the present application are further described below with reference to the accompanying drawings and specific embodiments, and however, the present application is not limited to the scope of the embodiments. In the examples, techniques or conditions, which are not specifically indicated, are performed according to techniques or conditions described in the literature of the art, or according to product instructions.

The reagents or instruments used herein, which are not indicated with manufacturers, are conventional products that are commercially available from formal sources.

### Example 1 Construction of a Lentiviral Vector Carrying a CYBB Gene

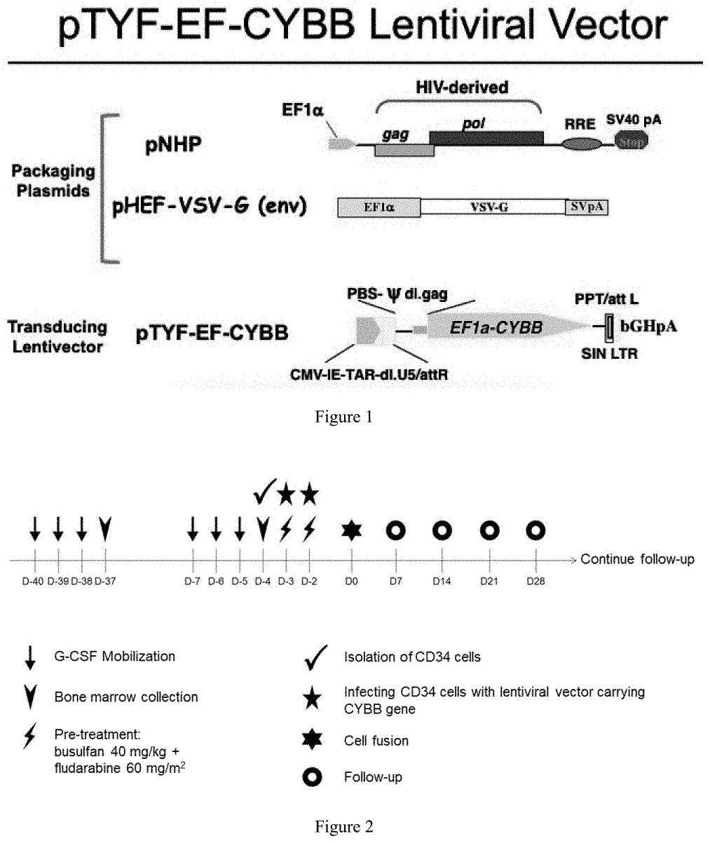

Normal CYBB gene sequence (amino acid sequence as shown in SEQ ID NO.2, and nucleic acid sequence as shown in SEQ ID NO.3) was synthesized via whole gene synthesis and ligated into TYF-EF1α lentiviral vector (NHP/TYF lentiviral vector system) through restriction enzyme digestion, behind a human EF1α (hEF1α) promoter sequence (nucleic acid sequence as shown in SEQ ID NO.1). The obtained product was identified by methods such as sequencing and double-digestion (cloned at BamHI site for 5′ and cloned at SpeI site for 3′, referring to NEB Manufacturer's recommendation for the reaction conditions) to obtain a properly linked lentiviral vector carrying CYBB gene under the hEF1α promoter. FIG. 1 shows the NHP/TYF lentiviral vector system, including virus packaging plasmids (NHP, EF-VSV-G) and a vector plasmid (pTYF-EF-CYBB). The packaging plasmids include pNHP and pHEF-VSV-G(env). pNHP expresses Gag-Pol protein, and pHEF-VSV-G expresses a coat protein. The gene delivery plasmid pTYF-EF carries a chimeric CMV-IE promoter at the 5′-end in combination with HIV-1 virus TAR-mutation U5 plus an attachment sequence at the right end (CMV-IE-TAR-dl.U5/attR), which is followed by a primer binding site (PBS) and a lentiviral vector packaging signal (psi) and a mutated gag sequence. EF1a-CYBB is followed by a mutated 3′LTR (self-inactivating SIN LTR), a polypurine track sequence (PPT), an attachment site at the left end (attL), and a bovine growth hormone polyA (bGHpA). See references [1]-[3] for details.

### Example 2 Lentivirus Packaging

A multi-plasmid packaging system was used in this example. The lentiviral vector carrying the CYBB gene was packaged into a complete lentivirus via 293T cells. The specific steps are:
(1) A 293T cell strain was cultured for 17-18 hours. Fresh DMEM containing 10% of FBS was added to the culture.
(2) DMEM, pNHP, pHEF-VSV-G and the lentiviral vector constructed in Example 1 were added to a sterile centrifuge tube successively, and vortexed.
(3) Superfect transduction reagent (QIAGEN) was added to the centrifuge tube and let stand at room temperature for 7-10 min.
(4) The lentiviral vector-Superfect mixture in the centrifuge tube was added dropwise to the 293T cells, vortexed, and incubated at 37° C. and 5% CO₂ for 4-5 hours.
(5) The cell culture medium was removed, the cells were rinsed, and culture medium was added to continue the incubation.
(6) The culture medium was returned to the 5% CO₂ incubator and incubated overnight, and then the transduction efficiency was observed with a fluorescence microscope.

### Example 3 Purification and Concentration of Lentivirus

The purification and concentration of lentivirus is performed as follows:

(1) Lentivirus Purification

The packaged lentivirus was centrifuged at 1000 g for 5 min to remove cell debris. The resulting supernatant was filtered using a 0.45 μm low protein binding filter, dispensed and stored at −80° C.

(2) Lentivirus Concentration

The lentivirus supernatant was added to a Centricon filter tube and centrifuged at 2500 g for 30 min. The filter tube was shaken and centrifuged at 400 g for 2 min. The concentrated virus was collected into a collection cup.

### Example 4 Transduction of Hematopoietic Stem Cells with Lentivirus

Hematopoietic stem cells (HSCs) were inoculated into a culture vessel. The concentrated lentivirus carrying the CYBB target gene was added, centrifuged at 100 g for 100 min, and incubated at 37° C. for 24 h. Medium containing stem cell growth factor was added and incubated for 2-3 days to obtain stem cells carrying normal CYBB genes.

### Example 5 Treatment of a Patient with X-Linked Chronic Granulomatous Disease (X-CGD) by Infecting CD34 Stem Cells with Lentivirus Carrying CYBB Gene (TYF-EF1a-CYBB)

FIG. 2 shows the treatment procedure.
(1) CD34 Stem cells of the patient was mobilized by granulocyte colony-stimulating factor (G-CSF). The patient was collected for peripheral blood or bone marrow twice, with the first collection performed on day 37 before the infusion and the second collection on day 4 before the infusion. Because CGD patients generally have a poor response to mobilization, two bone marrow collections are beneficial to obtain sufficient CD34 stem cells [Reference 4].
(2) In the laboratory on day 4 before the infusion, CD34-positive cells were isolated from both bone marrow or peripheral blood stem cells collected from the patient using Miltenyi CD34 beads and incubated overnight in HSC culture medium (Sigma Stemline II HSC expansion medium).
(3) On day 3 and 2 before the infusion, the patient was pre-treated with 40 mg/kg of body weight of busulfan and 60 mg/m² of body surface area of fludarabine, separately. According to the literature, proper pretreatment can effectively extend the survival of transgenic CD34 stem cells in patients [Reference 5].
(4) In the laboratory on day 3 and day 2 before the infusion, gene transduction was performed twice by infecting CD34 cells with lentivirus carrying the CYBB gene. Then the infected cells were incubated for one day.
(5) On the day of infusion, the cells were washed twice with normal saline containing 1% of human serum albumin protein, and suspended in normal saline containing of 2.5% human serum albumin protein, and infused back to the patient.
(6) After infusion to the patient, follow up was taken every week. The patient was collected for peripheral blood and measured for oxidase function, immune cell ratio and gene copy number.

### Result Analysis

The collected peripheral blood was stained with dihydrorhodamine 123 (DHR123), and CD14 and CD15 were additionally stained. Oxidase function in neutrophilic granulocytes and monocytes in peripheral blood was analyzed by flow cytometry. These two cells mainly use oxidase to perform immune functions. Dihydrorhodamine 123 is oxidized by hydrogen peroxide to rhodamine 123 which emits yellow-green fluorescence at 515 nm when excited by 488 nm laser. When dihydrorhodamine 123 is co-cultured with cells stimulated with phorbol ester (PMA), the fluorescence intensity represents the functional strength of oxidase [Reference 6].

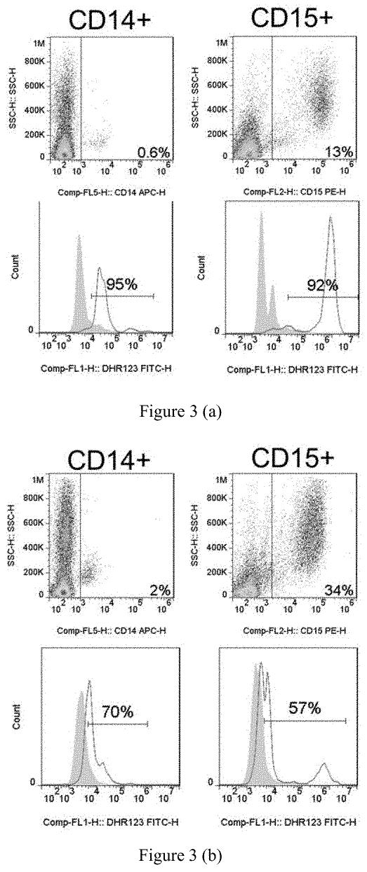

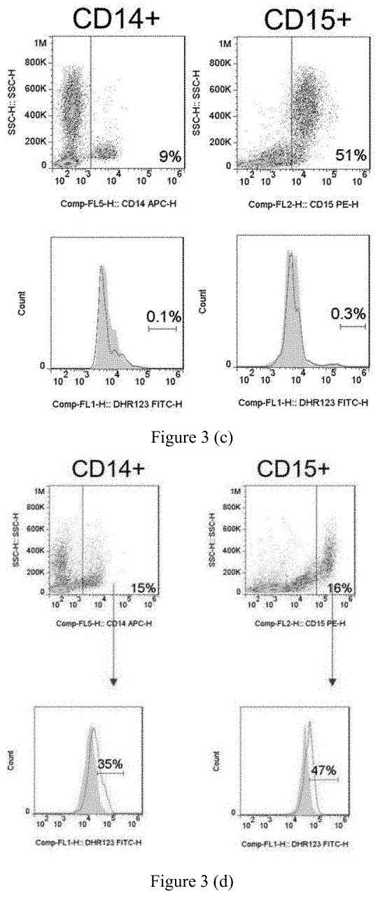

FIG. 3 (a) shows the analysis of peripheral blood from the patient's father who has a normal genotype. Both the proportions of monocytes (CD14+) and neutrophilic granulocytes (CD15+) expressing oxidase are greater than 90%. FIG. 3 (b) shows the analysis of peripheral blood from the patient's mother who has a heterozygous genotype. 70% of monocytes express oxidase and 57% of neutrophilic granulocytes express oxidase, both of which are slightly lower than the proportion of healthy people. FIG. 3 (c) shows the analysis of peripheral blood from the patient before infusion. It can be seen that the patient did not express oxidase at all before treatment. FIG. 3 (d) shows the analysis of peripheral blood from the patient on day 28 after treatment. 35% of monocytes and 47% of neutrophilic granulocytes in peripheral blood express oxidase, which are greatly improved compared with that before infusion.

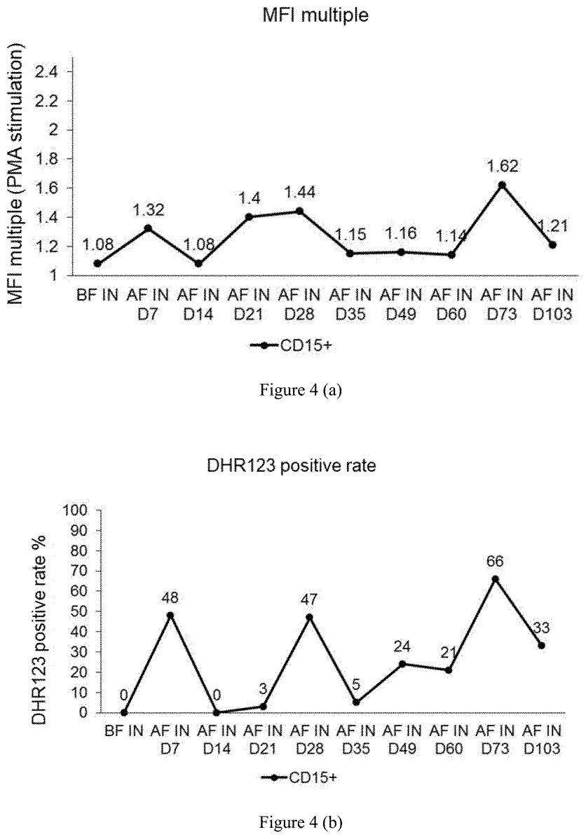

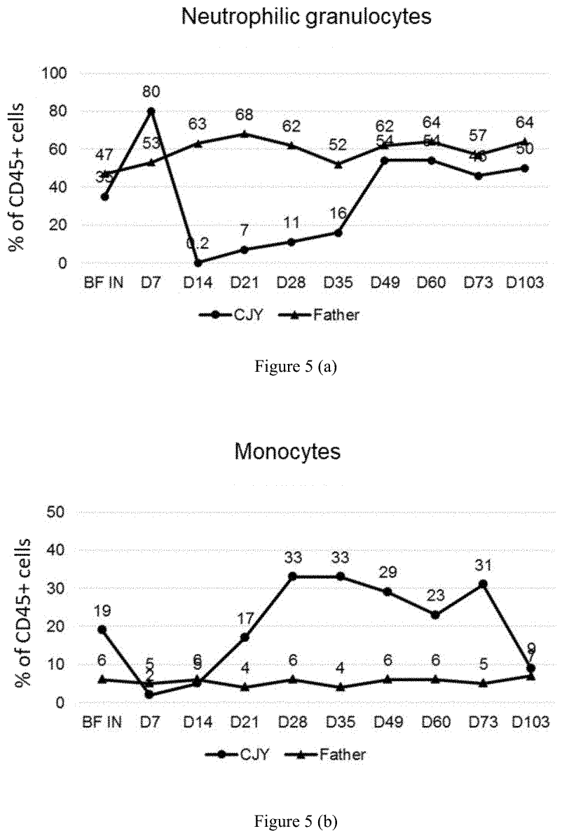

X-linked chronic granulomatous disease is mainly caused by oxidase deficiency in neutrophilic granulocytes. The functional strength and expression level of oxidase in neutrophilic granulocytes were observed weekly after the infusion. The results are shown in FIG. 5. FIG. 4 (a) shows the average fluorescence intensity multiple, that is the functional strength of oxidases in cells after stimulation, which is calculated by dividing the average fluorescence intensity of cells stimulated with phorbol ester (PMA) by the average fluorescence intensity of cells not stimulated with phorbol ester when rhodamine 123 was used for staining. Before the treatment, the patient's ratio was 1.08, suggesting that no oxidase response occurred in cells after received stimulation. After the treatment, the function of oxidase fluctuated with time, but improved overall, wherein day 28 and day 73 after the infusion showed the highest values, reaching 1.44-fold and 1.62-fold respectively. FIG. 4 (b) shows the proportion of neutrophilic granulocytes that emit fluorescence, that is, the proportion of neutrophilic granulocytes that express oxidase. The patient did not express any oxidase before the infusion. On day 28 and day 73 after the infusion, 47% and 66% of the patient's neutrophilic granulocytes expressed oxidase, respectively. The proportion of cells expressing oxidase has maintained above 20% since day 49 after the infusion (the patient received blood transfusion on day 7 after the infusion, which is not discussed herein).

Because the patient received myeloablation pretreatment before the infusion, the inventors monitored continuously the number of neutrophilic granulocytes and monocytes in CD45-positive cells in the patient after the infusion. As shown in FIG. 5 (a) on day 14 after infusion, the number of neutrophilic granulocytes reached the lowest point, accounting for only 0.2% of CD45-positive cells, which is obviously affected by the myeloablation pretreatment compared with the case in the normal genotype father whose neutrophilic granulocytes maintained at 60%. But after 14 days, the percentage of neutrophilic granulocytes gradually increased, and on day 49 after the infusion, to the normal proportion of healthy people and maintained till the latest follow-up, that is, day 103 after the infusion. As shown in FIG. 5 (b), the number of monocytes reached the lowest point on day 7 after the infusion, which accounts for only 2% of CD45-positive cells.

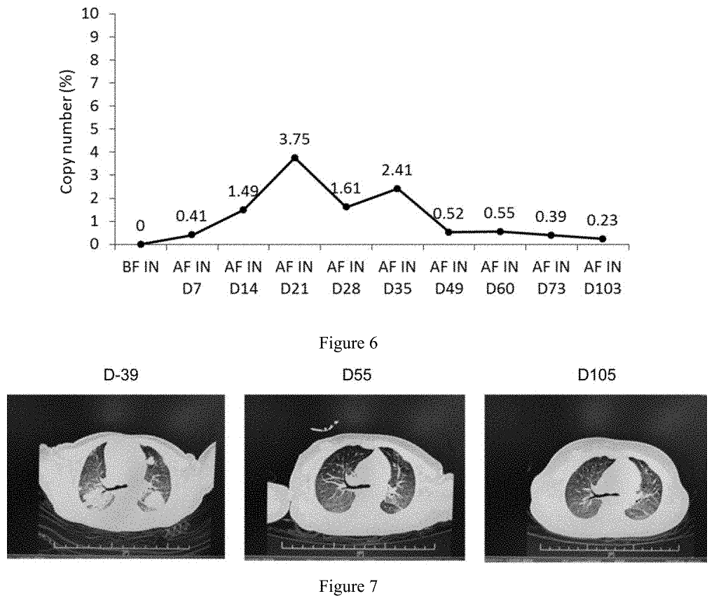

FIG. 6 shows the change of CYBB gene copy number in peripheral blood from the patient after infusion. On day 21 and day 35 after the infusion, the copy number reached 3.75% and 2.41%, respectively, which were the highest values monitored. It can be seen that CYBB gene was stably retained in peripheral blood cells. 0.23% of peripheral blood cells still carried CYBB gene even on day 103 after infusion.

In addition to molecular and cytological evidence, clinical symptoms of the patient were also improved significantly. FIG. 7 shows pulmonary CT scan images of the patient before and after infusion. On day 39 before the infusion, the patient had a severe infection in the lungs and received antifungal and antibacterial drugs as adjuvant therapy. On day 55 after the infusion, the pulmonary infection situation was improved significantly. On day 105 after the infusion, the pulmonary infection had relived and no drug support was required.

In summary, the lentiviral vector of the present disclosure achieves efficient delivery of CYBB gene under the initiation of the EF1α promoter. Lentivirus carrying the CYBB gene is used to transduce stem cells, which are together serve as a delivery vector for the treatment of CGD disease, such that CYBB gene expression is increased in differentiated or undifferentiated stem cells. Infection of CD34 stem cells with lentivirus carrying the CYBB gene has therapeutic potential for X-CGD.

### REFERENCES

[1] Chang, L.-J., V. Urlacher, T. Iwakuma, Y. Cui, and J. Zucali (1999). Efficacy and safety analyses of a recombinant human immunodeficiency virus derived vector system. Gene Therapy 6, 715-728.
[2] Cui, Y, T. Iwakuma and L.-J. Chang (1999). Contributions of viral splice sites and cis-regulatory elements to lentivirus vector functions. J Virol 73, 6171-6176.
[3] Iwakuma T., Y. Cui, and L.-J. Chang (1999). Self-inactivating lentiviral vectors with U3 and U5 modifications. Virology 261, 120-132.
[4] Sandhya R. Panch, Yu Ying Yau, Elizabeth M. Kang, Suk See De Ravin, Harry L. Malech and Susan F. Leitman (2014). Mobilization characteristics and strategies to improve hematopoietic progenitor cell mobilization and collection in patients with chronic granulomatous disease and severe combined immunodeficiency. Transfusion 55, 265-274.
[5] Manuel Grez, Janine Reichenbach, Joachim Schwable, Reinhard Seger, Mary C Dinauer, and Adrian J Thrasher. (2011) Gene Therapy of Chronic Granulomatous Disease: The Engraftment Dilemma. Molecular Therapy 19, 28-35.
[6] Yu Chen and Wolfgang G. Junger. (2012) Measurement of Oxidative Burst in Neutrophilic granulocytes. Methods in Molecular biology 844, 115-144.
[7] Douglas B. Kuhns et al. (2010) Residual NADPH Oxidase and Survival in Chronic Granulomatous Disease. The New England Journal of Medicine 363, 2600-2610.
[8] Danielle E. Arnold and Jennifer R. Heimall. (2017) A Review of Chronic Granulomatous Disease. Advances in Therapy 34, 2543-2557.
[9] Hyoung Jin Kang et al. (2011) Retroviral Gene Therapy for X-linked Chronic Granulomatous Disease: Results From Phase I/II Trial. Molecular Therapy 19, 2092-2101.
[10] De Ravin et al. (2017) CRISPR-Cas9 gene repair of hematopoietic stem cells from patients with X-linked chronic granulomatous disease. Science Translational Medicine 9, eaah3480.

The applicant states that detailed methods of the present application are demonstrated in the present application through the above embodiments, however, the present application is not limited to the above detailed methods, and does not mean that the present application must rely on the above detailed methods to implement. It should be apparent to those skilled in the art that, for any improvement of the present application, the equivalent replacement of the raw materials of the present application, the addition of auxiliary components, and the selection of specific modes, etc., will all fall within the protection scope and the disclosure scope of the present application.

1. A lentiviral vector, comprising a hEF1α promoter and CYBB that are organized in tandem.

2. The lentiviral vector according to claim 1, wherein the hEF1α promoter has a nucleic acid sequence as shown in SEQ ID NO.1.

3. The lentiviral vector according to claim 1, wherein the CYBB has an amino acid sequence as shown in SEQ ID NO. 2 and a nucleic acid sequence as shown in SEQ ID NO.3.

4. A lentivirus that is introduced with the lentiviral vector according to claim 1.

5. A host cell that is transduced with the lentivirus according to claim 4.

6. The host cell according to claim 5, wherein the host cell comprises a stem cell.

7. The host cell according to claim 6, wherein the stem cell comprises a hematopoietic stem cell.

8. A method for preparing a host cell according to claim 5, comprising the following steps:
(1) constructing a lentiviral vector comprising a hEF1α promoter and CYBB that are organized in tandem;
(2) performing lentivirus packaging by co-transducing the lentiviral vector obtained in step (1) and a packaging plasmid into a mammalian cell, to obtain a lentivirus; and
(3) transferring the lentivirus obtained in step (2) into the genome of a host cell.

9. The method according to claim 8, wherein the construction in step (1) is performed by inserting a hEF1α promoter and CYBB into a TYF lentiviral vector through restriction enzyme digestion.

10. The method according to claim 8, wherein the packaging plasmid in step (2) comprises pNHP and pHEF-VSVG.

11. The method according to claim 8, further comprising a step of purifying the lentivirus after step (2);
preferably, the purification is performed by filtering, centrifuging and concentrating the lentivirus.

12. The method according to claim 8, comprising the following steps:
(1) inserting a hEF1α promoter and CYBB into a TYF lentiviral vector through restriction enzyme digestion to construct a lentiviral vector;
(2) performing lentivirus packaging by co-transducing the lentiviral vector obtained in step (1) and packaging plasmids pNHP and pHEF-VSVG into a 293T cell to obtain a lentivirus, and purifying the lentivirus to obtain a concentrated lentivirus; and
(3) transforming the lentivirus obtained in step (2) into the genome of a hematopoietic stem cell.

13. A pharmaceutical composition, comprising the lentiviral vector according to claim 1.

14. The pharmaceutical composition according to claim 13, wherein the pharmaceutical composition further comprises any one or a combination of at least two of a group consisting of a pharmaceutically acceptable carrier, excipient and diluent.

15. (canceled)

16. A method for treating a disease, comprising administering to a patient in need thereof an effective amount of the lentiviral vector according to claim 1, wherein the disease comprises chronic granulomatosis.

17. The method according to claim 10, wherein the mammalian cell in step (2) comprises a 293T cell.

18. The method according to claim 11, further comprising a step of purifying the lentivirus after step (2).

19. The method according to claim 11, wherein the purification is performed by filtering, centrifuging and concentrating the lentivirus.

20. The method according to claim 11, wherein the host cell in step (3) comprises a stem cell.

21. The method according to claim 11, wherein the stem cell comprises a hematopoietic stem cell.


SEQUENCE LISTING (3 sequences)

SEQ ID NO: 1 - DNA, 1536 bp, Artificial Sequence
  nucleic acid sequence of hEF1alpha promoter
gctagcatgcctaggtcgaccaattctcatgtttgacagcttatcatcgataagctttggagctaagccagcaatggtagagggaagattctgcacgtcccttccaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactctagagcggccgcggaggccgaattccgtcgaggatccacc

SEQ ID NO: 2 - protein, 570 aa, Artificial Sequence
  amino acid sequence of CYBB
MGNWAVNEGLSIFVILVWLGLNVFLFVWYYRVYDIPPKFFYTRKLLGSALALARAPAACLNFNCMLILLPVCRNLLSFLRGSSACCSTRVRRQLDRNLTFHKMVAWMIALHSAIHTIAHLFNVEWCVNARVNNSDPYSVALSELGDRQNESYLNFARKRIKNPEGGLYLAVTLLAGITGVVITLCLILIITSSTKTIRRSYFEVFWYTHHLFVIFFIGLAIHGAERIVRGQTAESLAVHNITVCEQKISEWGKIKECPIPQFAGNPPMTWKWIVGPMFLYLCERLVRFWRSQQKVVITKVVTHPFKTIELQMKKKGFKMEVGQYIFVKCPKVSKLEWHPFTLTSAPEEDFFSIHIRIVGDWTEGLFNACGCDKQEFQDAWKLPKIAVDGPFGTASEDVFSYEVVMLVGAGIGVTPFASILKSVWYKYCNNATNLKLKKIYFYWLCRDTHAFEWFADLLQLLESQMQERNNAGFLSYNIYLTGWDESQANHFAVHHDEEKDVITGLKQKTLYGRPNWDNEFKTIASQHPNTRIGVFLCGPEALAETLSKQSISNSESGPRGVHFIFNKENF

SEQ ID NO: 3 - DNA, 1713 bp, Artificial Sequence
  nucleic acid sequence of CYBB
atggggaactgggctgtgaatgaggggctctccatttttgtcattctggtttggctggggttgaacgtcttcctctttgtctggtattaccgggtttatgatattccacctaagttcttttacacaagaaaacttcttgggtcagcactggcactggccagggcccctgcagcctgcctgaatttcaactgcatgctgattctcttgccagtctgtcgaaatctgctgtccttcctcaggggttccagtgcgtgctgctcaacaagagttcgaagacaactggacaggaatctcacctttcataaaatggtggcatggatgattgcacttcactctgcgattcacaccattgcacatctatttaatgtggaatggtgtgtgaatgcccgagtcaataattctgatccttattcagtagcactctctgaacttggagacaggcaaaatgaaagttatctcaattttgctcgaaagagaataaagaaccctgaaggaggcctgtacctggctgtgaccctgttggcaggcatcactggagttgtcatcacgctgtgcctcatattaattatcacttcctccaccaaaaccatccggaggtcttactttgaagtcttttggtacacacatcatctctttgtgatcttcttcattggccttgccatccatggagctgaacgaattgtacgtgggcagaccgcagagagtttggctgtgcataatataacagtttgtgaacaaaaaatctcagaatggggaaaaataaaggaatgcccaatccctcagtttgctggaaaccctcctatgacttggaaatggatagtgggtcccatgtttctgtatctctgtgagaggttggtgcggttttggcgatctcaacagaaggtggtcatcaccaaggtggtcactcaccctttcaaaaccatcgagctacagatgaagaagaaggggttcaaaatggaagtgggacaatacatttttgtcaagtgcccaaaggtgtccaagctggagtggcacccttttacactgacatccgcccctgaggaagacttctttagtatccatatccgcatcgttggggactggacagaggggctgttcaatgcttgtggctgtgataagcaggagtttcaagatgcgtggaaactacctaagatagcggttgatgggccctttggcactgccagtgaagatgtgttcagctatgaggtggtgatgttagtgggagcagggattggggtcacacccttcgcatccattctcaagtcagtctggtacaaatattgcaataacgccaccaatctgaagctcaaaaagatctacttctactggctgtgccgggacacacatgcctttgagtggtttgcagatctgctgcaactgctggagagccagatgcaggaaaggaacaatgccggcttcctcagctacaacatctacctcactggctgggatgagtctcaggccaatcactttgctgtgcaccatgatgaggagaaagatgtgatcacaggcctgaaacaaaagactttgtatggacggcccaactgggataatgaattcaagacaattgcaagtcaacaccctaataccagaataggagttttcctctgtggacctgaagccttggctgaaaccctgagtaaacaaagcatctccaactctgagtctggccctcggggagtgcatttcattttcaacaaggaaaacttctaa